(19) United States
(12) Patent Application Publication
(10) Pub. No.: US 2013/0211384 A1
(43) Pub. Date: Aug. 15, 2013

(54) THERAPEUTIC AGENT, COMPOSITION INCLUDING SAID AGENT, IMPLANTABLE DEVICE AND PROCESS FOR THE TREATMENT OF CERVICAL CANCER AND/OR FOR THE PREVENTION OF THE FORMATION OF NEOPLASMS IN CORRESPONDENCE OF THE CERVIX IN A HUMAN FEMALE GENITAL SYSTEM

(71) Applicant: Francesco Raspagliesi, Milan (IT)

(72) Inventor: Francesco Raspagliesi, Milan (IT)

(73) Assignee: FONDAZIONE IRCCS ISTITUTO NAZIONALE DEI TUMORI, Milan (IT)

(21) Appl. No.: 13/812,666

(22) PCT Filed: Jul. 28, 2010
(86) PCT No.: PCT/EP2010/060975

§ 371 Date: Mar. 29, 2013

(51) Int. Cl. A61M 31/00

(52) U.S. Cl. CPC A61M 31/002
USPC 604/515

(57) ABSTRACT

A medical composition including a chemotherapeutic medication or a targeted therapy medication is used in a human female patient for the treatment of a disease selected in the group comprising cervical cancer, cervical intraepithelial neoplasia (CIN), human papillomavirus (HPV) infection of the female genital system; the chemotherapeutic agent or targeted therapy medication is locally delivered directly to cervix of a female genital system by an implanted medical device including a stem to be implanted in the cervix. The stem has a drug carrying layer including the medical composition. A process for the treatment of the above diseases is also disclosed.

### TECHNICAL FIELD

This invention concerns a therapeutic agent, composition including said agent, implantable device and process for the treatment of cervical cancer and/or for the prevention of the formation of neoplasms in correspondence of the cervix in a human female genital system.

### BACKGROUND ART

As is known, carcinoma of the uterine cervix (CUC) is the second most frequent gynecological tumour in industrialised countries and the main cause of death in the female population in developing countries.

Despite the possibility of effective secondary prevention (pap test), tumours of the uterine cervix are the cause of around 4800 deaths every year in the United States, while in Italy around 3700 new cases and 1700 deaths are recorded every year.

Surgery represents the treatment of choice in the initial stages of the disease, that is to say when the tumour is confined into the cervix. In these stages, surgery can be followed by post-operative treatment based on various factors such as the presence of negative prognostic factors ((lymph node metastases).

In the more advanced stages, radiotherapy combined with concomitant chemotherapy is currently considered as the standard treatment.

Pre-operative (or neoadjuvant) chemotherapy for the treatment of locally advanced carcinoma of the cervix is an emergent and alternative therapeutic strategy and it has two different objectives:

a) to reduce the local extent of the disease in order to allow a radical surgery which would not otherwise be possible;
b) to cure any neoplastic emboli which have spread from the tumour to parts of the body remote from the primary tumour.

Recent meta-analyses of the randomized studies published to date on neoadjuvant chemotherapy have demonstrated a significant therapeutic benefit of this treatment, with a reduction in mortality risk of 36%.

Current practice is that of intravenously administer drugs. Intravenous administration of chemotherapeutic drugs leads to the onset of toxic systemic effects (which differ according to the drugs used) such as: nausea, vomiting, alopecia, nephrotoxicity, neurotoxicity, myelotoxicity, cardiotoxicity, gastrointestinal toxicity, cutaneous toxicity, pulmonary fibrosis.

It also known from U.S. Pat. No. 6,982,091 to administer chemotherapeutic agents or inhibitors of membrane efflux systems to the vagina using e.g. a vaginal tampon, vaginal ring, vaginal strip, vaginal capsule, vaginal tablet, vaginal bioadhesive tablet, vaginal pessary, vaginal cup or vaginal sponge incorporated with a transmucosal composition. This solution too has caused toxic side effects and requires use of a relatively high dosage of therapeutic agent.

### SUMMARY OF THE INVENTION

In this situation it is an object of the invention to offer a solution effective to treat cervix cancer and to effectively treat pre-cancer formations as well.

It is a further object that of offering a technical solution suitable to minimize systemic toxicity.

It is an additional object of the invention to offer a solution where the amount of therapeutic agent delivered to the patient is sensibly reduced while increasing effectiveness in the treatment of the cervix pathology.

It is an auxiliary object of the invention a solution which can be implemented without use of complex devices.

Furthermore, it is an object of the invention that of that of rendering available a solution where the therapy can be easily tailored to the specific patient.

One or more of the above objects are substantially reached by a therapeutic agent, a composition, a medical device and a process according to the appended claims.

Aspects of the invention are disclosed here below.

In a 1^{st} aspect it is provided a therapeutic agent selected in the group comprising a chemotherapeutic medication and a targeted therapy medication, for the treatment of a disease selected in the group comprising:
cervical cancer,
cervical intraepithelial neoplasia (CIN),
   human papillomavirus (HPV) infection of the female genital system.

The therapeutic agent is to be used for the treatment of the above illnesses in a human female patient, wherein the chemotherapeutic agent or targeted therapy medication is to be locally delivered directly to cervix of a female genital system.

In a 2^{nd} aspect a therapeutic agent is provided for the preparation of a medical composition for use in the treatment of a disease in a human female patient, the disease being selected in the group comprising:
cervical cancer,
cervical intraepithelial neoplasia (CIN),
   human papillomavirus (HPV) infection of the female genital system, wherein:
   wherein the therapeutic agent is a chemotherapeutic medication and/or a targeted therapy medication and wherein the medical composition is to be locally delivered directly to cervix of a female genital system.

In a 3^{rd} aspect according to any one of the preceding aspects the therapeutic agent or the medical composition is to be locally delivered directly to cervix of a female genital system through a medical device implanted in correspondence of the cervix.

In a 4^{th} aspect according to any one of the preceding aspects the therapeutic agent or the medical composition is contained in a portion of said implanted medical device, said portion being designed to come into direct contact with the cervix tissues, said therapeutic agent or medical composition being locally delivered directly to the tissues of the cervix through: diffusion through pores of said portion, and/or convection through pores of said portion, and/or biodegradation of said portion.

In a 5^{th} aspect according to any one of the preceding aspects the therapeutic agent or the medical composition is delivered in a sustained release manner, either continuously or in a pulsed manner.

In a 6^{th} aspect according to any one of the preceding aspects the therapeutic agent or the medical composition is to be delivered for a time period comprised between 1 and 360 days, optionally between 1 and 3 weeks.

In a 7^{th} aspect according to any one of the preceding aspects the therapeutic agent or the medical composition is located in said portion which is in the form of a coating of the medical device, said coating comprising a multi-layer structure

In a 8^{th} aspect according to the 7^{th} aspect wherein said therapeutic agent or said medical composition is included in a plurality of layers of the multi-layer structure.

In a 9^{th} aspect according to anyone of the preceding aspects, the agent or the medical composition is applied onto a surface of the implanted medical device.

In a 10^{th} aspect according to any one of the preceding aspects the therapeutic agent comprises a chemotherapeutic medicament.

In an 11^{th} aspect according to the 10^{th} aspect the chemotherapeutic medicament includes one or more selected in the group including: Cisplatin, Carboplatin, Taxol, Taxotere, Topotecan, Irinotecan, Adriamycin, Gemcitabine, Bleomycin, Ifosfamide, Vinorelbine, Fluorouracil, VP 16, Metrotrexate, Mitomycin C, Vincristine, Vinblastine.

In a 12^{th} aspect according to the 11^{th} aspect the therapeutic agent comprises Taxol and wherein Taxol is released at a dose from 0.1 to 140 mg (milligrams) per week.

In a 13^{th} aspect according to the 12^{th} aspect Taxol is delivered at a dose of from 1 to 30 mg (milligrams) per week.

In a 14^{th} aspect according to any one of the preceding aspects from the 10^{th} to the 13^{th} the chemotherapeutic medicament comprises Cisplatin and wherein Cisplatin is released at a dose from 0.1 to 80 mg (milligrams) per week.

In a 15^{th} aspect according to the 14^{th} aspect wherein Cisplatin is delivered at a dose of from 1 to 40 mg (milligrams) per week.

In a 16^{th} aspect according to any one of the preceding aspects wherein the therapeutic agent comprises a targeted therapy medication having at least one of the functions selected in the group comprising: inhibition of neo-angiogenesis and tumor vascularization, inhibition of tumor cell proliferation, induction of programmed tumor cell death, eradication of HPV infection responsible for carcinogenesis.

In a 17^{th} aspect according to any one of the preceding aspects wherein the therapeutic agent comprises a targeted therapy medication providing inhibition of neo-angiogenesis function, said targeted therapy medication acting on pathways responsible for tumor vascularization through one selected in the group comprising: antibodies directed against VEGF, inhibitors of specific receptor tyrosine kinases, inhibitors of intracellular signal transductors.

In an 18^{th} aspect according to any one of the preceding aspects wherein the therapeutic agent comprises a targeted therapy medication providing inhibition of tumor cell proliferation, said targeted therapy medication acting on pathways responsible for growth factors through one selected in the group comprising: antibodies directed against specific receptor tyrosine kinase EGFR, inhibitors of EGFR signal transduction.

In a 19^{th} aspect according to any one of the preceding aspects wherein the is therapeutic agent comprises a targeted therapy medication providing induction of programmed tumor cell death, said targeted therapy medication acting on inhibition of mechanisms protecting from senescence and death to induce tumor cell apoptosis.

In a 20^{th} aspect according to any one of the preceding aspects wherein the therapeutic agent comprises a targeted therapy medication providing eradication of HPV infection responsible for cancerogenesis said targeted therapy medication targeting Human Papilloma Virus by vaccination to induce specific anti-viral immune response or by antiviral therapy to eliminate integrated HPV.

In a 21^{st} aspect according to any one of the preceding aspects said agent or said medical composition is in crystalline form optionally comprising crystals having an average crystalline size comprised between 0.1 μm and 100 μm

In a 22^{nd} aspect according to any one of the preceding aspects said agent or said medical composition is in crystalline form optionally comprising crystals having an average crystalline size between 1 and 10 μm.

In a 23^{rd} aspect according to any one of the preceding aspects from the 2^{nd} to the 22^{nd} said medical composition comprises excipients.

In a 24^{th} aspect it is provided a medical composition for use in the treatment of a disease in a human female patient, the disease being selected in the group comprising:
cervical cancer,
cervical intraepithelial neoplasia (CIN),
   human papillomavirus (HPV) infection of the female genital system, wherein:
   wherein the medical composition comprises a therapeutic agent according to any one of the preceding aspects and
   wherein the medical composition is to be locally delivered directly to cervix of a female genital system.

In a 25^{th} aspect according to the 24^{th} aspect the medical composition includes a polymer matrix wherein the therapeutic agent is hosted in the polymer matrix, wherein the agent and the matrix are designed to define a sustained release dosage form capable of causing a delivery of the agent for a time period comprised between 1 and 360 days, optionally for a time period comprised between 1 and 3 weeks.

In a 26^{th} aspect according to the 25^{th} aspect the polymer matrix is made of a biodegradable polymer.

In a 27^{th} aspect according to the 26^{th} aspect the therapeutic agent is dispersed into the polymer matrix and/or inserted into pores of the polymer matrix.

In a 28^{th} aspect according to any one of the preceding aspects from 25^{th} to 27^{th} said polymer matrix comprises a biodegradable polymer.

In a 29^{th} aspect according to any one of the preceding aspects from 25^{th} to 28^{th} said polymer matrix comprises a polymer selected in the group including: styrene-isobutylene-styrene (SIBS), a polyanhydride copolymer, Poly (bis(P-corboxyphenoxy)propane-sebacic acid, poly(D, L lactic-co-glycolic acid).

In a 30^{th} aspect it is provided an implantable medical device comprising: a stem designed to positioned into the cervix of a human female genital system; at least a drug carrying portion associated to at least a surface of the stem, wherein the drug carrying portion includes a therapeutic agent according to any one of the preceding aspects or a medical composition according to any one of the preceding aspects.

In a 31^{st} aspect according to the 30^{th} aspect the implantable medical device comprises an axial blocking element associated to the stem, the axial blocking member axially blocking the stem relative to the cervix when the stem is inserted into the same cervix.

In a 32^{nd} aspect according to the 31^{st} aspect the axial blocking member comprises an expandable member which can be moved from a first configuration wherein the expandable member is in a collapsed state to a second configuration wherein the expandable member is in an expanded state, in said second configuration the expandable member being radially bigger than the stem and than the same expandable member in said first configuration.

In a 33^{rd} aspect according to the 32^{nd} aspect the implantable medical device comprises a further axial blocking member axially apart from said axial blocking member.

In a 34^{th} aspect according to the 33^{rd} aspect the further axial blocking member comprises a further expandable member which can be moved from a first configuration, wherein the further expandable member is in a collapsed state, to a second configuration, wherein the further expandable member is in an expanded state, in said second configuration the further expandable member being radially bigger than the stem and than the same further expandable member in said first configuration.

In a 35^{th} aspect according to any one of preceding aspects from the 30^{th} to 34^{th} said expandable member comprises an expandable balloon, e.g. an inflatable balloon.

In a 36^{th} aspect according to any one of preceding aspects from the 30^{th} to 35^{th} said axial blocking member comprises at least a portion of the stem which can radially expand.

In a 37^{th} aspect according to the 36^{th} aspect said portion which can radially expand comprises hydrophilic material which, when placed in contact with body fluids, can absorb a portion of said body fluids and at least radially increase in volume.

In a 38^{th} aspect according to the 36^{th} or 37^{th} aspect said portion which can radially expand comprises an elastically deformable material which, when constricted, can take a radially compact size and which, when released, can radially expand. For instance the portion can be made totally or in part of elastic material and can include longitudinal plies to facilitate contraction and expansion.

In a 39^{th} aspect according to the 36^{th} or 37^{th} or 38^{th} aspect said portion which can radially expand comprises a shape memory alloy portion which, when subject to a thermal treatment, can radially expand.

In a 40^{th} aspect according to any one of aspects from the 30^{th} to the 39^{th}, the axial blocking member comprises a plate element located at a caudal end of said stem.

In a 41^{st} aspect according to the 40^{th} aspect said plate element comprises a curved concave side facing said stem and a convex side opposite said concave side.

In a 42^{nd} aspect according to any one of aspects from the 30^{th} to the 41^{st} the axial blocking member is located at a proximal end of said stem while the further axial blocking member (if present) is located at a proximal end of said stem.

In a 43^{rd} aspect according to any one of aspects from the 30^{th} to the 42^{nd} said drug carrying portion comprises a drug carrying layer covering at least a portion of the surface of the stem.

In a 44^{th} aspect according to the 43^{rd} aspect, said drug carrying layer covers a surface portion located at least in correspondence of a caudal region of the stem.

In a 45^{th} aspect according to the 43^{rd} aspect, said drug carrying layer covers a surface portion located at least in correspondence of a proximal region of the stem.

In a 46^{th} aspect according to the 43^{rd} or 44^{th} or 45^{th} aspect, said drug carrying layer covers a surface portion located at least in correspondence of the side surface of the stem, i.e. in correspondence of the cylindrical or frustoconical lateral surface of the stem.

In a 47^{th} aspect according to any one of aspects from the 43^{rd} to the 46^{th} one of the axial blocking members includes a plate element and said drug carrying layer or drug carrying portion covers a portion of said plate element, optionally wherein said drug carrying layer covers the surface of the concave side of said plate element.

In a 48^{th} aspect according to any one of aspects from the 30^{th} to the 47^{th} comprising a plurality of mutually overlapping drug carrying layers, each of said drug carrying layers including at least a therapeutic agent according to any one of aspects from the 1^{st} to the 23^{rd} or a medical composition according to any one of aspects from the 2^{nd} to the 29^{th}.

In a 49^{th} aspect according to any one of aspects from the 30^{th} to the 48^{th} an to intermediary layer is positioned between each of said drug carrying layers, said intermediary layer not including drugs.

In a 50^{th} aspect according to any one of aspects from the 30^{th} to the 49^{th} at least a number of said drug carrying layers comprises a first medicament composition and at least a number of said drug carrying layers comprise a second medicament composition different from said first medicament composition.

In a 51^{st} aspect according to any one of aspects from the 30^{th} to the 50^{th} the implantable medical device comprises a fluid supply channel connecting one or both said expandable member and said further expandable member with an external fluid supply for allowing said expandable member to move from said respective first configuration to said respective second configuration.

In a 52^{nd} aspect according to any one of aspects from the 30^{th} to the 51^{st} the implantable medical device comprises a discharge channel extending axially along the stem and creating a fluid communication between an area external to said inflatable element and an area external to said further inflatable member.

In a 53^{rd} aspect according to the 52^{nd} aspect said discharge channel is extending parallel to said fluid supply channel.

In a 54^{th} aspect it is disclosed a process for treatment of one illness of a human female patient, the illness being selected in the group comprising: cervical cancer, cervical intraepithelial neoplasia (CIN), human papillomavirus (HPV) infection of the female genital system, the process comprising the step of locally delivering directly to cervix of a female genital system at least one of the therapeutic agents according to any one of the preceding aspects from the 1^{st} to the 23^{rd} and/or at least one of the medical compositions according to the preceding aspects from the 2^{nd} to the 29^{th}.

In a 55^{th} aspect according to the 54^{th} aspect said local delivery is achieved by implanting in correspondence of the cervix of a human female patient a medical device according to anyone of the preceding aspects from the 30^{th} to the 53^{rd}.

In a 56^{th} aspect according to the 54^{th} or 55^{th} aspect said local delivery comprises a local delivery of said therapeutic agent at least for a period comprised between 1 and 360 days, optionally for a period between 1 and 3 weeks.

In a 57^{th} aspect according to the 54^{th} or 55^{th} or 56^{th} aspect the process further includes combining said local delivery with a systemic delivery of a therapeutic agent selected in the group comprising a chemotherapeutic medication and/or a targeted therapy medication.

In a 58^{th} aspect according to any one of preceding aspects from the 54th to the 57^{th}/implanting comprises: inserting the device through the vagina, positioning the stem of the device in correspondence of the cervix, leaving the device inside the female genital system.

In a 59^{th} aspect according to the preceding aspect the device is removed after a period of 1 to three weeks and a new device of the type according to any one of aspects from the 30^{th} to the 53^{rd} inserted. The cycle is repeated a plurality of times, e.g. 3 to 5 times.

In a 59^{th} aspect according to the 58^{th} aspect the device is left inside the cervix until complete biodegradation and a new device of the type according to any one of aspects from the 30^{th} to the 53^{rd} inserted. The cycle is repeated a plurality of times, e.g. 3 to 5 times.

### BRIEF DESCRIPTION OF THE DRAWINGS

Aspects of the invention will be described here below with reference to the appended drawings, which are provided by way of non-limiting example, in which:

FIG. 1 is a schematic view of a first example of an implantable medical device, with the device implanted in correspondence of the cervix of a female genital system,

FIG. 2 is an enlarged view of the device of FIG. 1,

FIG. 3 is a cross section along line III-III of the stem of the device shown in FIG. 2;

FIG. 4 is a schematic view of a second example of an implantable medical device, with the device implanted in correspondence of the cervix of a female genital system,

FIG. 5 is an enlarged view of the device of FIG. 4,

FIG. 5A shows the particular 50 of FIG. 5,

FIG. 6 is a cross section along line VI-VI of the stem of the device shown in FIG. 5;

FIG. 7 is a schematic view of a third example of an implantable medical device, with the device implanted in correspondence of the cervix of a female genital system,

FIG. 8A is an enlarged view of the device of FIG. 7,

FIG. 8B is a cross section along line VIII-VIII of the stem of the device shown in FIG. 8A;

FIG. 9 is a schematic view of a fourth example of an implantable medical device, with the device inserted in a positioning cannula which can be used to insert the device in correspondence of the cervix,

FIG. 10 is a view of the device of FIG. 9 during extraction from the cannula,

FIG. 11 is an interrupted cross section along line XI-XI of the stem of the device shown in FIG. 2;

FIG. 12 shows a schematic diagram of the release rate versus time in an example of the invention.

### DETAILED DESCRIPTION

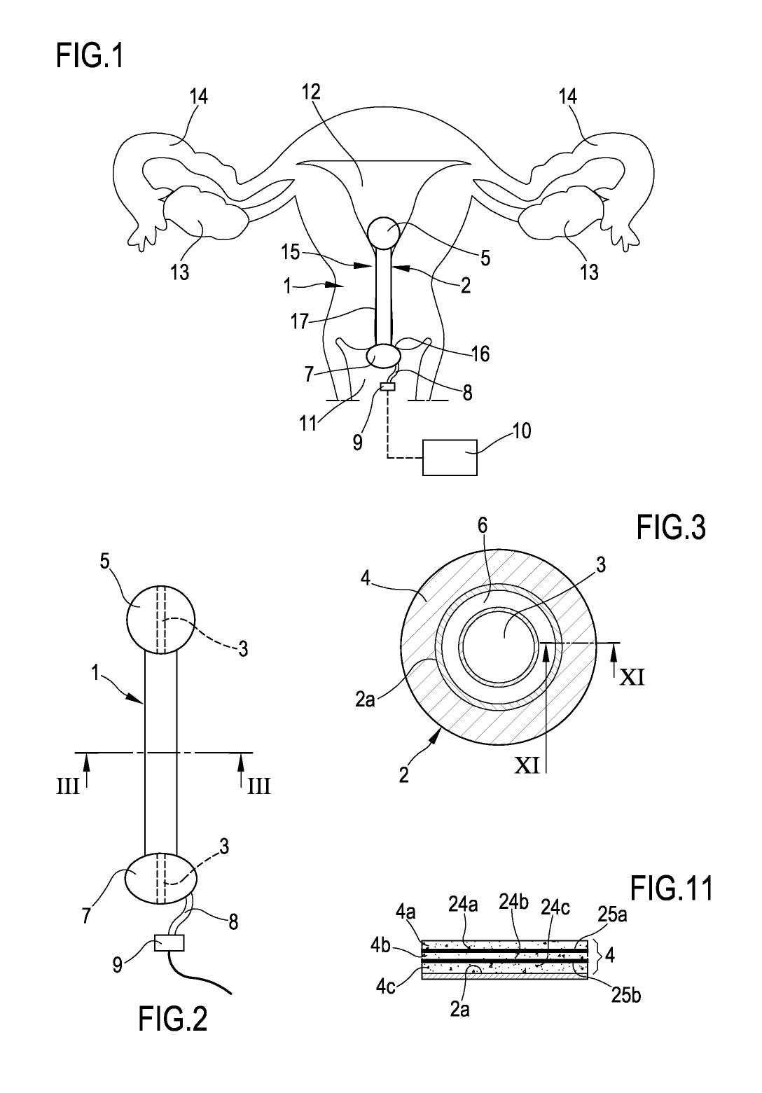

With reference to FIGS. 1,4 and 7, it is schematically represented a human female genital system which includes the vagina 11, the uterus 12, and the cervix (or neck of the uterus) 15 which is the lower narrow portion of the uterus where it joins with the top end of the vagina. The cervix 15 comprises the endocervical canal 17, which is about 3 to 5 cm long, and the ectocervix 16, which is the portion of the cervix projecting into the vagina. Although the cervix varies widely in length and width, it takes a substantially cylindrical or conical overall shape, as shown in the drawings. The enclosed drawings also show the ovaries 13 and the fallopian tubes 14.

The present invention relates to medical compositions, devices and processes for the effective treatment of cervical cancer or of cervical tissues with the aim of preventing formation of neoplasms in correspondence of the cervix.

In accordance with aspects of the invention, and with reference to FIGS. 1 through 7, an implantable medical device 1 for use in the treatment of cervical tissues is disclosed. The device 1 is designed and shaped to be stably positioned into the cervix of a human female genital system; the stem 2 can for instance present an elongated substantially cylindrical or substantially frustoconical shape: the length of the stem can be in the range between 2 and 6 cm, optionally between 2 and 4 cm and the diameter can be between 2 and 4 mm. The stem can be tubular and include a through cavity 6. At least a drug carrying portion 4 is associated to at least a surface 2a of the stem 2; the drug carrying portion comprises one or more therapeutic agents and/or medical compositions of the type herein after disclosed.

In order to axially block the stem 2 with respect to the cervix 15 the implantable medical device comprises at least one axial blocking member. As it will be further explained the axial blocking member can be part of the stem or can include one or more additional elements engaged to the stem; in any case, the axial blocking member axially blocks the stem relative to the cervix when the stem is inserted into the same cervix. In a first example shown in FIGS. 1-5, it provided an axial blocking member 5 which comprises an expandable member: the expandable member can be moved from a first configuration wherein the expandable member is in a collapsed state to a second configuration wherein the expandable member is in an expanded state; in the second configuration, the expandable member is radially bigger than the stem 2 and also radially bigger than the radial size of the same expandable member in the first configuration. In practice, when the expandable member is in the first configuration (not shown in the figures), the expandable member has a radial size substantially same or inferior compared to the diameter of the stem so that the device can be inserted through the cervix. Once the stem is in proper position, the expandable member can be moved to the second configuration where it presents a radial size of e.g. around from 3 mm to 6 mm or even higher so as to interfere with the inner wall of the uterus and avoid extraction of the implanted medical device. In accordance with an option, as shown in FIGS. 1-5, the device 1 also comprises a further axial blocking member 7 axially apart from the above described axial blocking member 5. Under a constructional point of view, also the further axial blocking member can be in the form of another expandable member which can be moved from a first configuration, wherein the further expandable member is in a collapsed state, to a second configuration, wherein the further expandable member is in an expanded state; in the second configuration the further expandable member is radially bigger than the stem and also radially bigger than the same further expandable member in the first configuration. Once the stem is in proper position, the further expandable member can be moved to the second configuration where it presents a radial size of e.g. around from 3 mm to 6 mm or even higher so as to interfere with the inner wall of vagina and avoid axial movement of the implanted medical device towards the uterus. The axial blocking member 5 is at a proximal end of the stem while the further axial blocking member 7 is at a caudal end of the stem; note that in the context of the present disclosure, proximal end refers to the portion of the stem which in use is closest to the uterus, while distal or caudal end refers to the portion of the stem which in use is closest to the vagina. In the example of FIGS. 1 and 2, each of the expandable member 5 and the further expandable member 7 comprises a respective inflatable balloon which can be inflated by supplying a fluid inside the inflatable balloon. For instance a fluid supply line 8 can extend from the inside of each balloon to a fluid supply source 10. A valve 9 can be present on the fluid supply line to close the line when the necessary fluid has been supplied. For removing fluid from the balloons, the valve can be opened to cause fluid evacuation and deflation of the balloons. Note that, instead of a fluid supply line of the type described, balloons can also be inflated by injecting a fluid through a disposable line connected to the balloon: in this case each balloon could be provided with a respective check valve operative in correspondence of an inlet port on the balloon so that once the balloon is inflated and the supply conduit separated from the balloon the check valve would prevent evacuation of fluid from the balloon.

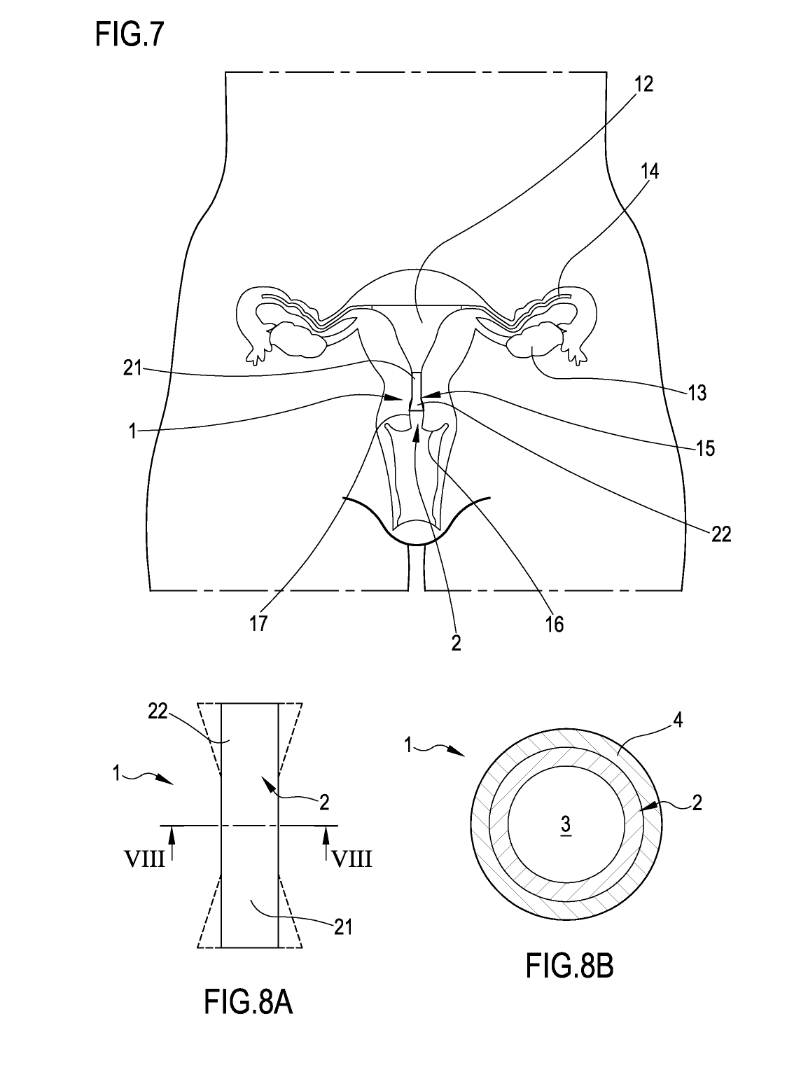

As an alternative, or in addition to the above described balloons, the axial blocking member may be part of the stem. In other words, the stem 2 may comprise at least a portion 21, e.g. an axial segment, which can radially expand in order to anchor the medical device with respect to the cervix. In FIGS. 7 and 8 a device 1 having two portions 21 and 22 of the stem which can radially increase in size (see dashed lines schematically representing the change in size of said portions) is represented. For instance a portion 21, 22 of the stem can be made or comprise hydrophilic material which, when placed in contact with body fluids, can absorb part of said body fluids and increase in radial volume. Alternatively, the stem can include a radially expandable portion including a shape memory alloy (SMA), such as copper-zinc-aluminium-nickel, copper-aluminium-nickel, and nickel-titanium, zinc-copper-gold-iron. SMAs alloys “remembers” their original, cold-forged shape and return to that shape after being deformed by applying an appropriate change in temperature. In practice, the stem 2 may include portion or portions which can be heated or cooled after installation so as to appropriately change geometry (see portions 21 and 22) and form respective blocking members.

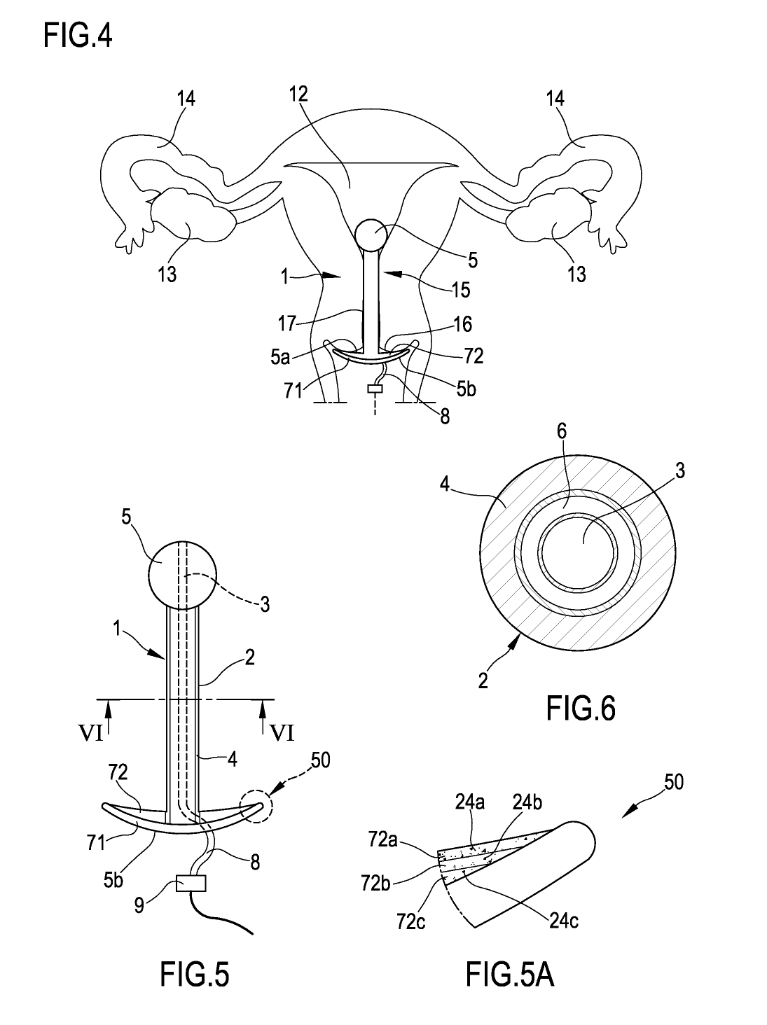

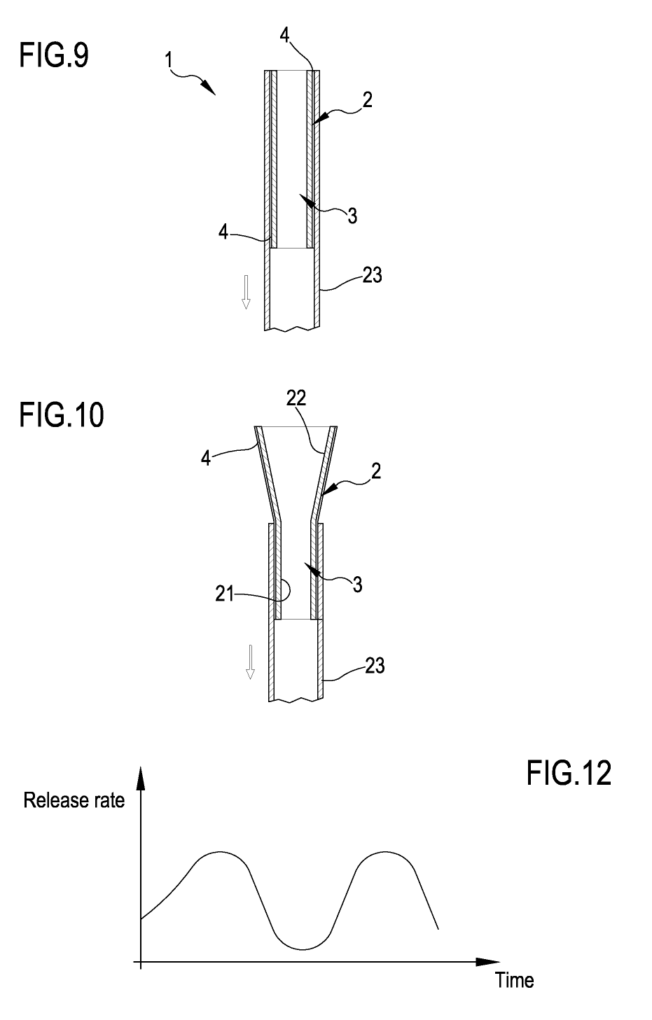

According to a further alternative, the stem (or one or more stem portions) can be made of an elastic material and be shaped such as to be radially compressible. When radially constricted, the stem or the stem portions can take a radially compact size and, when released, the stem or stem portions can radially expand. For instance an axial segment 21, 22 or the entire stem 2 can be made of a material which can be compacted, e.g. constricted by a tubular cannula 23, and which when released by the cannula would spontaneously tend to return to an expanded state thus creating an interference with the cervix wall and blocking the stem in the cervix. This solution is schematically shown in FIGS. 9 and 10. Finally, according to a further alternative shown in FIGS. 4-6, the axial blocking member may comprise a plate element 71 located at a caudal end of said stem. In the example shown in FIGS. 4 and 5 the plate element is at a caudal end, while one of the above described expandable members (e.g. an inflatable balloon 5) is present at a proximal end of the stem 2 (i.e. on the side of the uterus). The plate element of the device of FIGS. 4 and 5 comprises a curved concave side 5a facing said stem 2 and a convex side 5b opposite said concave side.

As shown in the figures, the implantable medical device can also include a discharge channel 3 (the discharge channel can be present in any one of the above described embodiments) extending axially along the stem and creating a fluid communication between two axially opposed areas which in use should communicate with the vagina and with the uterus respectively in order to allow discharge of fluids from the uterus when the device 1 is installed in the cervix. For instance, the discharge channel can be coaxial to the stem and can go through the inflatable elements and the plate (where the plate and or inflatable elements are present). Of course the channel 3 can also be obtained on the periphery of the stem by properly shaping the stem contour: for instance the stem cross section could present a peripheral indent defining the channel 3.

The drug carrying portion 4, which may be in the form of a drug carrying layer, covers at least a portion of the free surface of the stem. The drug carrying layer 4 covers a surface portion located on the side surface of the stem. For instance the drug carrying layer 4 may cover the entire side surface of the stem, or a portion thereof for instance a portion of the stem side surface located in correspondence of a caudal region of the stem or of a proximal region of the stem. In any case the portion 4 is positioned to come into direct contact with the is tissues of the cervix 15. In case of devices 1 including the plate element 71 as one of the axial blocking members, the drug carrying portion includes a the drug carrying layer 72 covering a portion of said plate element. More in detail, the drug carrying layer 72 covers the surface of the concave side 5a of said plate element which is basically designed to abut, in use, against the ectocervix 16. The drug carrying portion or drug carrying layer comprises a matrix and a therapeutic agent dispersed or inserted in the matrix so as to cause a sustained release of the therapeutic agent which can release from the matrix by diffusion through the matrix and/or by virtue of the matrix biodegradation; the matrix can be a polymer matrix as further described herein below. As the drug carrying layer is in direct contact with the tissues of the cervix, the tissues are effectively treated as substantially all the therapeutic agent is conveyed exactly where desired. Going in further detail it should be noted that the drug carrying layer 4 and/or 72 can be in one layer only or in multiple layers. For instance, the drug carrying layer present on the surface of the stem and/or the drug carrying layer present on the surface of the plate element may include a plurality of mutually overlapping layers 4a, 4b, 4c and 72a, 72b, 72c respectively. An example is shown in FIG. 11, which is a section along trace XI-XI of FIG. 3 of a portion of the stem 2; another example is represented in FIG. 5A which shows a possible variant of the layer 72. Each of said layers 4a, 4b, 4c and/or 72a, 72b, 72c may include at least a medical composition or a therapeutic agent 24a, 24b, 24c of the type disclosed below. Different layers may include the same or a different medicament composition/therapeutic agent. Moreover, the same medicament composition or therapeutic agent may be included in different layers at different concentrations in order to appropriately tailor the agent release rate.

In some solutions, it may be possible to have an intermediary layer 25a, 25b without drugs or active agents positioned between each of said drug carrying layers 4a, 4b, 4c. In case the intermediary layer is made of bioeredible material, it is possible to have a pulsed release of the therapeutic agent included in the multi layer structure, as schematically shown in FIG. 12.

In accordance with one aspect, drug carrying portions or drug carrying layer include a therapeutic agent or a medical composition having said therapeutic agent. The therapeutic agent can be or can include a chemotherapeutic medication and/or a targeted therapy medication. This type of agents present in the drug carrying layer or portion 4 of the implanted medical device, as above described, demonstrated to be effective for the treatment of cervical cancer (squamous cell carcinoma, adenocarcinoma), cervical intraepithelial neoplasia (CIN), and of human papillomavirus (HPV) infection of the human female genital system. The efficiency is accentuated by the local delivery directly to cervix of a female genital system as the medical composition is contained in a drug carrying portion, which can e.g. be in the form of a layer coating, designed to come into direct contact with the cervix tissues so that the medical composition or the therapeutic agent is locally delivered to the tissues of the cervix by diffusion through the coating layer/layers or drug carrying portion/portions or by virtue of the progressive degradation of the drug carrying portions (e.g. is the matrix is made of a biodegradable polymer).

In certain cases, the medical composition or the therapeutic agent can be designed such that the agent is delivered in a sustained release manner, either continuously or in a pulsed manner, e.g. for a time period comprised between 1 and 360 days, optionally between 1 and 3 weeks. More specifically, one or more of the layers forming the drug carrying portion can be constituted by a dry crystalline form of the agent (with basically no polymer matrix): when the agent is brought into contact with the tissues of the cervix, body liquids cause the crystalline structure to undergo a phase change thereby progressively dissolving the crystalline structure and causing a sustained release effect of the agent.

Alternatively, the therapeutic agent, either alone or in a composition including other agents and/or one or more excipients, may be inserted or dispersed into a carrier such as a polymer matrix. The polymer matrix can be porous and/or biodegradable: in any case the degree of porosity and the biodegradability of the polymer matrix in combination with the state of aggregation of the agent (crystalline or not) and with the agent concentration in the matrix determine the release rate of the agent and the duration of the sustained release effect. The polymer matrix wherein the agent is inserted or dispersed may comprise a polymer selected in the group including: styrene-isobutylene-styrene (SIBS), a polyanhydride copolymer, Poly (bis(P-corboxyphenoxy)propane-sebacic acid, poly(D, L lactic-co-glycolic acid).

In accordance with a further aspect, the medical composition or the therapeutic agent, for instance one of the below identified chemotherapeutic agents (see section “Therapeutic agents used for local delivery”), can be in crystalline form. More in detail, the crystals can have an average crystalline size comprised between 0.1 μm and 100 μm, so as to provide an additional sustained release effect. In some examples, for instance in case the agent is Taxol or Cisplatin, the average size of the crystals is in the range between 1 and 10 μm.

As mentioned, the medical composition or therapeutic agent is part of a sustained release dosage form which is designed such as to cause a delivery of the agent for a time period comprised between 1 and 360 days. In certain examples, the sustained release dosage form is designed such as to cause a delivery of the agent for a time period comprised between 1 and 3 weeks. The sustained release effect is provided by several factors, which can be tailored according to the needs:
the nature and concentration of the agent dispersed or embedded into the polymer matrix,
the nature of the polymer matrix,
the state of aggregation of the agent (crystalline form or not) and the size of the crystals.

Thus, proper selection of the above variables allows to obtain the desired sustained release effect.

### Therapeutic Agents Used for Local Delivery

The following agents (chemotherapeutic medications or targeted therapy medications) can be used for local delivery using any one of the devices 1 above described.

The chemotherapeutic medications can comprises one or more selected in the group including Cisplatin, Carboplatin, Taxol, Taxotere, Topotecan, Irinotecan, Adriamycin, Gemcitabine, Bleomycin, Ifosfamide, Vinorelbine, Fluorouracil, VP 16, Metrotrexate, Mitomycin C, Vincristine, Vinblastine.

In one example, the drug carrying portion (which can be in the form of a layer applied as above described to the stem surface) includes Taxol or a medical composition including Taxol and is designed to release Taxol at a dose from 0.1 to 140 mg (milligrams) per week, optionally at a dose of from 1 to 30 mg (milligrams) per week. The drug carrying portion is designed to provide a sustained release of the agent for a period up to one year, more frequently for a period up to 3 weeks. Alternatively, the drug carrying portion (which can be in the form of a layer applied as above described to the stem surface) includes Cisplatin or a medical composition including Cisplatin and is designed to release Cisplatin at a dose from 0.1 to 80 mg (milligrams) per week, optionally at a dose of from 1 to 40 mg (milligrams) per week. The drug carrying portion is designed to provide a sustained release of the agent for a period up to one year, more frequently for a period up to 3 weeks.

In a further alternative, the therapeutic agent can comprise a targeted therapy medication. Targeted therapy defines therapeutic strategies aimed to modify the pathways through which cells regulate their interaction with the external environment and their reproductive functions. Modifications include either downmodulation or activation of receptors, enzymes, proteins or mediators involved in cellular response to growth factors, senescence, hypoxia, immune response, cell-cell interaction and extracellular matrix interface. Since tumor-genesis occurs through the further alterations of most of these interactions, targeting specific molecules in their regulatory pathways will control tumor cell growth and metastasis, while sparing normal cells. The targeted therapy medication used according to aspects of the invention has at least one of the following functions:

A) inhibition of neo-angiogenesis and tumor vascularization,

B) inhibition of tumor cell proliferation,

C) induction of programmed tumor cell death,

D) eradication of HPV infection responsible for carcinogenesis.

### A. Inhibition of Neo-Angiogenesis

The pathways responsible for tumor vascularization will be inhibited through:
1. antibodies directed against VEGF (e.g. Bevacizumab)
2. inhibitors of specific receptor tyrosine kinesis such as VEGFR and/or bFGFR (e.g. Sunitinib, Sorafenib)
3. inhibitors of intracellular signal transductors (e.g. Imatinib, Nilotinib)

### B. Inhibition of Tumor Cell Proliferation

The pathways of response to growth factors will be inhibited through:
1. antibodies directed against specific receptor tyrosine kinase EGFR (e.g. Cetuximab)
2. inhibitors of EGFR signal transduction (e.g. Gefitinib, Erlotinib)

### C. Induction of Programmed Tumor Cell Death

Inhibition of mechanisms protecting from senescence and death will induce tumor cell apoptosis. Proteosoma inhibitors such as Bortezomib target these pathways.

### D. Eradication of HPV Infection Responsible for Tumorgenesis

Targeting Human Papilloma Virus can be done in a preventive or in a curative way
1. vaccination to induce specific anti-viral immune response
2. antiviral therapy to eliminate integrated HPV (Lopinavir)

Also in case of use of targeted therapy medications, the drug carrying portion 4 is designed to provide a sustained release of the medication for a period up to one year, more frequently for a period up to 3 weeks.

### Examples

Cetuximab administration using the device 1 is of particular interest for treatment of the above pathologies.

VEGFR overexpression is associated with a poor prognosis in several solid tumors including cervical cancer in which higher VEGF levels correlate with higher stages and increased risk of lymphnodes metastasis. In addition, emerging data suggest that HPV directly stimulates VEGF production through upregulation of the E6 oncoprotein. Bevacizumab delivered with the device 1 can be used for treatment of advanced cervical cancer; Bevacizumab delivered with the device 1 can be used alone and/or in combination with chemotherapy (cisplatin, paclitaxel and topotecan) which can be delivered either with device 1 or intravenously. Optionally radiotherapy can be added.

Taxol or Cisplatin can be locally delivered with the device 1. The local delivery of Taxol and/or Cisplatin can be effectively combined with an IV delivery of the same drug or drugs or with the IV delivery of targeted therapy medicaments.

In all cases, the amount of drug to be delivered can be reduced obtaining very promising therapeutic results while reducing the overall systemic toxicity.

### Procedures

In accordance with aspects of the invention, the treatment of a disease selected in the group comprising cervical cancer, cervical intraepithelial neoplasia (CIN), human papillomavirus (HPV) infection of the female genital system, using one of below procedures:
I. locally deliver directly to the cervix tissues one of the above chemotherapeutic medicaments using one of the described implanted devices, or
II. locally deliver directly to the cervix tissues one of the above targeted therapy medicaments, or
III. locally deliver directly to the cervix tissues a plurality of the above chemotherapeutic medicaments using one of the described implanted devices (for instance a different chemotherapeutic agent can be placed in each layer of a multi-layer drug carrying portion 4), or
IV. locally deliver directly to the cervix tissues a plurality of the above targeted therapy medications using one of the described implanted devices (for instance a targeted therapy medication can be placed in each layer of a multi-layer drug carrying portion 4), or
V. locally deliver directly to the cervix tissues one or more of the above targeted therapy medications in combination with one or more of the above chemotherapeutic agents using one of the described implanted devices (for instance a targeted therapy medication can be placed in one layer of a multi-layer drug carrying portion 4, while a chemotherapeutic agent can be placed in another layer of the multi-layer portion 4), or
VI. combine a systemic delivery (e.g. via IV infusion) of a solution including one of the above identified chemotherapeutic agents with the local delivery according to one of above points from Ito V.
VII. combine a systemic delivery (e.g. via IV infusion) of a solution including one of the above identified targeted therapy medications with the local delivery according to one of above points from 1 to 5.

### Exemplifying Methods of Manufacture

Under a constructional point of view the drug carrying portion can be, for example, obtained adopting one of below manufacturing processes.

According to one example, a mixture of a polymer solution with the therapeutic agent can be prepared and then the stem can be dipped in the solution to receive the mixture on a surface thereof, then extracted and then dried. The process can be repeated multiple times to create a multilayer structure. If one wants to create layers with different compositions, then different mixtures have to be prepared.

Alternatively a dry powder including the therapeutic agent can be dusted onto the surface of the stem. Adhesives can be used on the stem surface or excipients cam be mixed with the therapeutic agent to facilitate adhesion of the agent to the stem surface. A single or a multi layer structure can be created. Of course other manufacturing processes can be used.

1-50. (canceled)

51. A method of treatment of one illness of a human female patient,
the illness being selected from the group consisting of:
cervical cancer,
cervical intraepithelial neoplasia (CIN), and
human papillomavirus (HPV) infection of the female genital system,
by locally delivering, directly to cervix of a female genital system, at least one therapeutic agent selected from the group consisting of:
a chemotherapeutic medication, and
a targeted therapy medication,
wherein locally delivering comprises implanting a medical device in correspondence of the cervix, the medical device having:
a stem positioned into the cervix of a human female genital system and
at least a drug carrying portion associated to at least a surface of the stem, the therapeutic agent being contained in the drug carrying portion which comes into direct contact with the cervix tissues.

52. A method according to claim 51 wherein said therapeutic agent is locally delivered directly to the tissues of the cervix via one or more selected from the group consisting of: diffusion through pores of said drug carrying portion, convection through pores of said drug carrying portion, biodegradation of said drug carrying portion.

53. A method according to claim 51, wherein said therapeutic agent is locally delivered directly to the tissues of the cervix in a sustained release manner at least for a period comprised between 1 and 360 days.

54. A method according to claim 53, wherein said therapeutic agent is locally delivered directly to the tissues of the cervix at least for a period comprised between 1 and 3 weeks.

55. A method according claim 51 further comprising combining said local delivery with a systemic delivery of a therapeutic agent selected from the group consisting of a chemotherapeutic medication and a targeted therapy medication.

56. A method according to claim 51 wherein locally delivering comprises a cycle comprising:
inserting the device through the vagina,
positioning the stem of the device in correspondence of the cervix, and
leaving the device inside the female genital system for at least one day.

57. A method according to claim 56, the cycle further comprising removing the device after a period of 1 to three weeks and repeating a plurality of times said cycle using for each cycle a new device of the same type.

58. A method according to claim 56, wherein positioning the stem comprises axially blocking the stem relative to the cervix when the stem is inserted into the same cervix and wherein axially blocking comprises adopting an axial blocking member at a proximal end of the stem, the proximal end being the portion of the stem which in use is closest to the uterus.

59. A method according to claim 56, wherein said drug carrying portion covers the entire side surface of the stem, and is positioned to come into direct contact with the tissues of the cervix.

60. A method according to claim 51 wherein the therapeutic agent comprises a chemotherapeutic medicament which comprises one or more selected from the group consisting of: Cisplatin, Carboplatin, Taxol, Taxotere, Topotecan, Irinotecan, Adriamycin, Gemcitabine, Bleomycin, Ifosfamide, Vinorelbine, Fluorouracil, VP 16, Metrotrexate, Mitomycin C, Vincristine, Vinblastine.

61. A method according to claim 51 wherein the therapeutic agent comprises Taxol and wherein Taxol is released at a dose from 0.1 to 140 mg (milligrams) per week.

62. A method according to claim 51 wherein the therapeutic agent comprises Cisplatin and wherein Cisplatin is released at a dose from 0.1 to 80 mg (milligrams) per week.

63. A method according to claim 51 wherein the therapeutic agent comprises one selected from the group consisting of:
a targeted therapy medication having inhibition of neo-angiogenesis and tumor vascularization,
a targeted therapy medication having inhibition of tumor cell proliferation,
a targeted therapy medication having induction of programmed tumor cell death, and
a targeted therapy medication having eradication of HPV infection responsible for carcinogenesis.

64. A method according to claim 63 comprising a targeted therapy medication providing inhibition of neo-angiogenesis function, said targeted therapy medication acting on pathways responsible for tumor vascularization through one selected from the group consisting of: antibodies directed against VEGF, inhibitors of specific receptor tyrosine kinases, and inhibitors of intracellular signal transductors.

65. A method according to claim 63 comprising a targeted therapy medication providing inhibition of tumor cell proliferation, said targeted therapy medication acting on pathways responsible for growth factors through one selected from the group consisting of: antibodies directed against specific receptor tyrosine kinase EGFR, and inhibitors of EGFR signal transduction.

66. A method according to claim 63 comprising a targeted therapy medication providing induction of programmed tumor cell death, said targeted therapy medication acting on inhibition of mechanisms protecting from senescence and death to induce tumor cell apoptosis.

67. A method according to claim 63 comprising a targeted therapy medication providing eradication of HPV infection responsible for cancerogenesis said targeted therapy medication targeting Human Papilloma Virus by vaccination to induce specific anti-viral immune response or by antiviral therapy to eliminate integrated HPV.

68. A method according to claim 63, wherein said therapeutic agent is in crystalline form comprising crystals having an average crystalline size comprised between 0.1 μm and 100 μm.

69. A method according to claim 51 wherein the therapeutic agent is hosted in a polymer matrix, wherein the therapeutic agent and the matrix are designed to define a sustained release dosage form capable of causing a delivery of the agent in a sustained release manner for a time period comprised between 1 and 360 days, further wherein the polymer matrix comprises a biodegradable polymer matrix with said agent being dispersed into the polymer matrix and/or being inserted into pores of the polymer matrix.

70. A method of treatment of one illness of a human female patient,
the illness being selected from the group consisting of:
cervical cancer, and
cervical intraepithelial neoplasia (CIN),
by locally delivering, directly to the cervix of a female genital system, at least one therapeutic agent comprising Taxol,
wherein locally delivering comprises implanting a medical device in correspondence of the cervix, the medical device having:
a stem positioned into the cervix of a human female genital system and
at least a drug carrying portion associated to at least a surface of the stem, the therapeutic agent being contained in the drug carrying portion which comes into direct contact with the cervix tissues,
wherein Taxol is released at a dose of from 1 to 30 mg (milligrams) per week.

71. A method of treatment of one illness of a human female patient,
the illness being selected from the group consisting of:
cervical cancer,
cervical intraepithelial neoplasia (CIN), and
human papillomavirus (HPV) infection of the female genital system,
by locally delivering, directly to the cervix of a female genital system, at least one therapeutic agent comprising Cisplatin,
wherein locally delivering comprises implanting a medical device in correspondence of the cervix, the medical device having:
a stem positioned into the cervix of a human female genital system and
at least a drug carrying portion associated to at least a surface of the stem, the therapeutic agent being contained in the drug carrying portion which comes into direct contact with the cervix tissues,
and wherein Cisplatin is released from 1 to 40 mg (milligrams) per week.
